(19) United States
(12) Patent Application Publication
(10) Pub. No.: US 2016/0015321 A1
(43) Pub. Date: Jan. 21, 2016

(54) ORAL HEALTH MONITORING AND MANAGEMENT

(71) Applicant: A. T. Still University, Mesa, AZ (US)

(72) Inventor: Shervin Tony Hashemian, Scottsdale, AZ (US)

(21) Appl. No.: 14/799,514

(22) Filed: Jul. 14, 2015

Related U.S. Application Data
(60) Provisional application No. 62/024,714, filed on Jul. 15, 2014.

(51) Int. Cl. A61B 5/00; A61B 10/00

(52) U.S. Cl. CPC A61B 5/682; A61B 5/0017; A61B 10/0051

(57) ABSTRACT

Innovative oral health monitoring technologies, which may be embodied in various apparatuses, systems, and methods, provide enhanced patient sample pH testing and other health data. The technologies may involve an oral health monitoring apparatus, which may include a mouthpiece housing, a pH sensor, and a wireless communication interface. The mouthpiece housing may be fitted to a mouth of a patient and may, by way of the pH sensor, capture saliva pH measurements. The technologies may involve wirelessly transmitting the measurements to a server for health condition correlation analyses, visual report generation, and subsequent display on a user device.

### CROSS-REFERENCE TO RELATED APPLICATION

The present application claims the priority benefit of U.S. provisional patent application No. 62/024,417 filed Jul. 15, 2014, the entire disclosure of which is incorporated herein by reference.

### BACKGROUND OF THE INVENTION

1. Technical Field of the Invention

The present disclosure relates to oral health monitoring and management. More particularly, the present disclosure relates to technologies that monitor and analyze saliva pH levels.

2. Description of the Related Art

The oral cavity is the gateway and window into the health of the human body. Signs of nutritional deficiencies, general infections, systemic diseases, and other conditions that affect the entire body may first become apparent in the oral cavity (e.g., lesions, and other oral problems). Saliva in particular plays a significant role in maintaining oral health. Like many substances (e.g., urine, blood, sweat, tears, skin oils, other bodily fluids), saliva may be characterized by its pH (e.g., a measure of how acidic or basic a substance is). A pH level of a substance may change, however, in response to certain circumstances. Such changes in acidity or alkalinity of a patient's bodily fluids may be a risk factor or may otherwise indicate a change that may have an effect on health.

A low PH level in the oral cavity has a strong correlation to tooth decay and other systemic conditions. Increase of intraoral acidity usually brings forth dental disease, caries, and erosion. Ongoing studies indicate that saliva may also be useful for detecting various cancers, including breast and oral cancers, heart disease, diabetes, periodontal and gum diseases, viral hepatitis, and other conditions. Saliva is already used for rapid HIV testing, for example. In the dental field, medical or dental professionals commonly measure patient sample pH through pH level test strips. The use of such test strips limits the amount and relevancy of any data that may be collected.

Thus, there is a need in the art for innovative and cost-effective oral health monitoring technologies that, by providing enhanced patient sample pH testing and other valuable health data, improve health and quality of life metrics for the global health community.

### SUMMARY OF THE CLAIMED INVENTION

Innovative oral health monitoring technologies are claimed herein.

In a first claimed embodiment, an oral health monitoring apparatus includes a mouthpiece housing, a pH sensor, and a wireless communication interface. The pH sensor captures pH measurements of saliva in a mouth of a patient. The wireless communication interface sends the captured pH measurements to a server over a wireless communication network.

In a second claimed embodiment, a system for monitoring oral health includes a monitoring device that captures a plurality of pH measurements of saliva in a mouth of a patient. The system also includes a server that receives the pH measurements from the monitoring device over a wireless communication network.

In a third claimed embodiment, a computer-implemented method of monitoring oral health includes receiving pH measurements at a server. The measurements are pH measurements of saliva in a mouth of a patient. The method includes storing the received pH measurements in memory of the server. The method also includes receiving measurements regarding health indicators for the patient via a communication interface of the server. The method further includes executing instructions stored in memory of the server, at which point the server generates a visual display of the pH measurements over time.

### BRIEF DESCRIPTION OF THE DRAWINGS

FIG. 1 illustrates an exemplary network environment in which embodiments of the oral health monitoring technologies disclosed herein may be implemented.

FIG. 2 illustrates an exemplary oral health monitoring apparatus.

FIG. 3 illustrates an exemplary oral health monitoring apparatus worn in a mouth of a patient.

FIG. 4 illustrates an exemplary pH sensor that may be implemented in the context of the oral health monitoring apparatus described herein.

FIG. 5 illustrates an exemplary mold that may be used to create a mouthpiece housing.

FIG. 6 illustrates an exemplary operational flow of an exemplary oral health monitoring system.

FIG. 7 illustrates an exemplary computer-implemented method of monitoring oral health.

FIG. 8 illustrates an exemplary system for implementing a computing device.

### DETAILED DESCRIPTION

Innovative oral health monitoring technologies are provided. The technologies, which may be embodied in various apparatuses, systems, and methods, provide enhanced patient sample pH testing and other valuable health data. In various embodiments, the technologies may involve software applications (e.g., smartphone applications) that operate in conjunction with one or more sensors (e.g., a pH sensor) to capture time-sensitive health data and wirelessly communicate the health data to a server in compliance with Health Insurance Portability and Accountability Act (HIPAA) regulations. The technologies may be used by medical or dental professionals to educate patients, assist in preventive treatment planning, and properly select medical or dental materials in order to initiate positive changes in a patient's oral hygiene.

An oral health monitoring apparatus may include a mouthpiece housing, a pH sensor, and a wireless communication interface. The mouthpiece housing may be fitted to a mouth of a patient, thus enabling the patient to comfortably wear the apparatus even for long durations. The pH sensor, which may be disposed inside or outside the mouthpiece housing, may capture pH measurements of saliva in the mouth of the patient. The pH measurements may be captured over a predetermined time interval. The wireless communication interface, which may likewise be disposed inside or outside the mouthpiece housing, sends the captured pH measurements to a server over a wireless communication network.

An oral health monitoring system may include a monitoring device that captures a plurality of pH measurements of saliva in a mouth of a patient. The system may also include a server communicatively coupled to the monitoring device over a communications network. The communications network may be a wireless communications network, either wholly or in part (e.g., where the communications network is a series of linked subnetworks). The server may receive the pH measurements from the monitoring device over the communication network. The server may also generate a visual display of the pH measurements over time, which may include generating visual display data representing the pH measurements over time. The visual display data may be rendered and displayed directly by the server or it may be transmitted to a separate and distinct device (e.g., a user device, such as a tablet or smartphone) that may render and provide the visual display through a user interface.

The monitoring device, which may include a mouthpiece housing, may capture the plurality of pH measurements of saliva by way of a pH sensor. The monitoring device may capture the pH measurements over a predetermined time interval. The pH sensor may be disposed inside or outside the mouthpiece housing. The mouthpiece housing may be fitted to the mouth of the patient. The monitoring device may also include a wireless communication interface that communicates with the server over the communications network. Like the pH sensor, the wireless communication interface may be disposed inside or outside the mouthpiece housing.

A computer-implemented method of monitoring oral health may include receiving pH measurements at a server. The measurements may be pH measurements of saliva in a mouth of a patient and may have been captured over a predetermined time interval by a monitoring device. The pH measurements may be received by the server over a wireless communication network and may be received from a monitoring device. The method may include storing the received pH measurements in memory of the server. The method may also include receiving measurements regarding health indicators for the patient via a communication interface of the server. Each health indicator measurement may be associated with a time. The method may further include executing instructions stored in memory of the server. Upon execution of the instructions by a processor of the server, the method may include generating a visual display of the pH measurements over time. The visual display may further include the health indicator measurements over time.

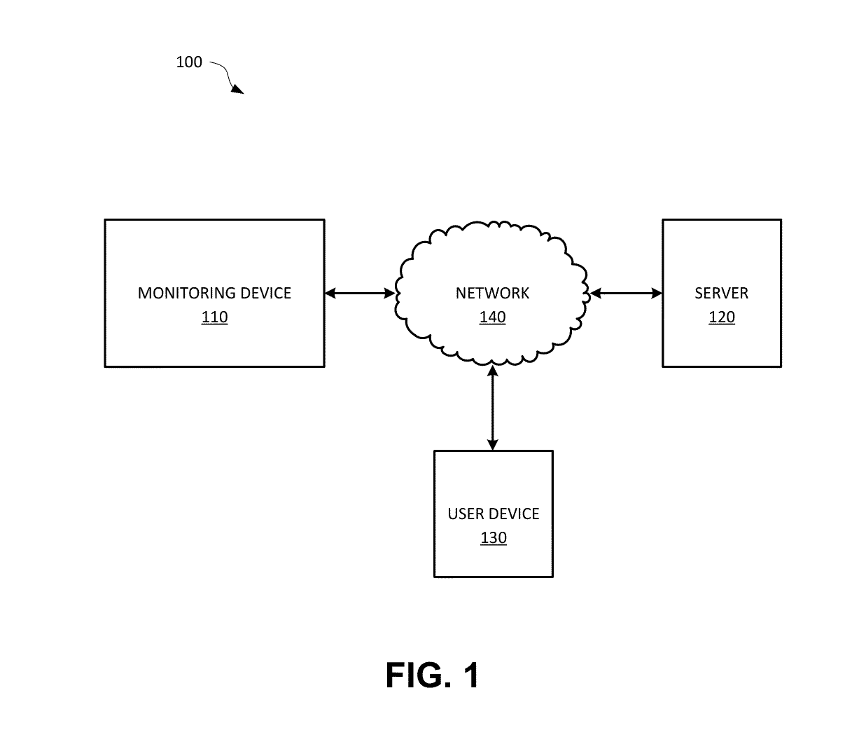

FIG. 1 illustrates an exemplary network environment 100 in which embodiments of the oral health monitoring technologies disclosed herein may be implemented. The oral health monitoring technologies may be implemented by way of various embodiments (e.g., apparatuses, systems, methods, or computer-readable non-transitory storage media) within a variety of network contexts. Accordingly, although certain drawings have been presented for the purpose of illustration, they should not be construed as limited to the precise forms disclosed. By way of an example, where FIG. 1 illustrates an exemplary network environment that includes various network devices, such as a monitoring device, a server, and a user device, persons of ordinary skill in the art will readily appreciate that any given network environment may, in practice, include a host of other network devices, including routers, switches, gateways, bridges, and other devices. Notwithstanding the network architecture shown, it should be understood that the described embodiment is exemplary and that, in other possible embodiments, the technology may be utilized in connection with numerous other network arrangements.

As illustrated in FIG. 1, exemplary environment 100 may include a monitoring device 110, a server 120, and a user device 130, all of which may be communicatively coupled by a communications network 140 either directly or through any number of intermediate servers, gateways, or other network devices.

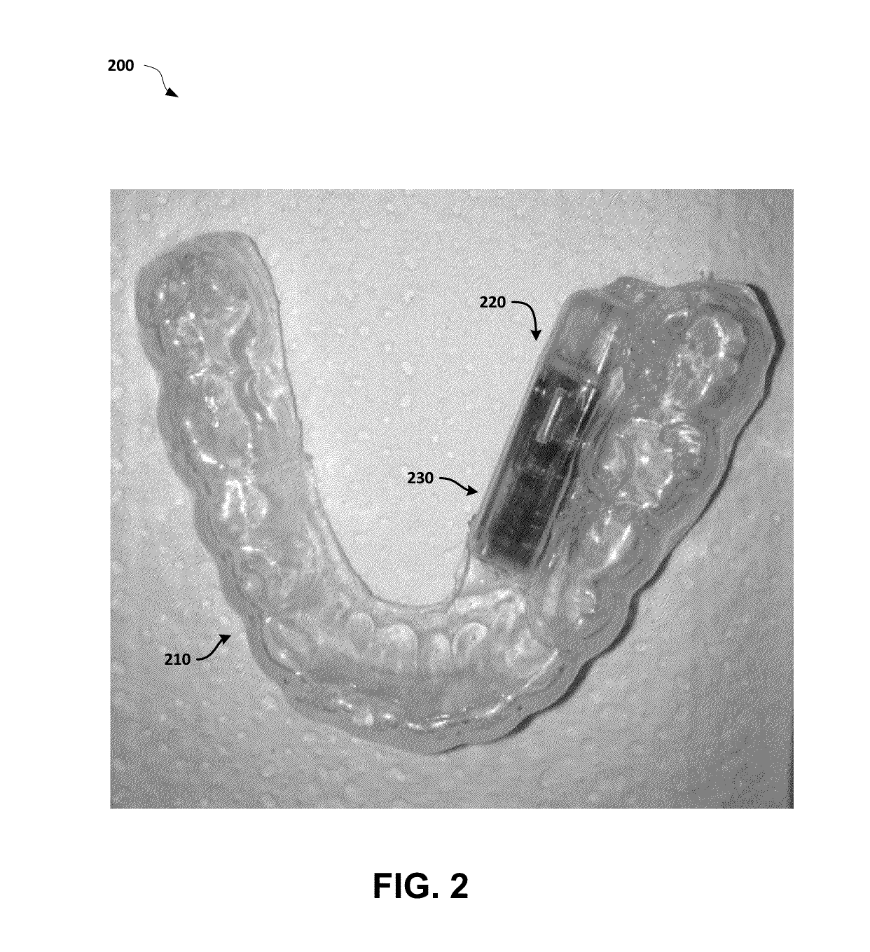

Monitoring device 110, an exemplary embodiment of which is described in greater detail with reference to FIGS. 2 through 5, may be a computing device that includes a processor, memory, and a wireless communications interface or other network interface. Monitoring device 110 may be worn by a patient. Monitoring device 110 may monitor a pH level of saliva in a mouth of the patient and may send a plurality of pH measurements to server 120 over network 140.

Server 120 may be a computing device that includes a processor, memory, and a network interface (e.g., a server, desktop computer, workstation, laptop, smartphone, tablet, electronic reader, a smart watch, various types of mobile devices, or other type of computing device). Server 120 may be communicatively coupled to network 140 by way of the network interface. Server 120 may a web server, a resource server, a database server, an application server, a domain controller, or any other type of server.

User device 130 may be any computing device that includes a processor, memory, and a network interface (e.g., a desktop computer, workstation, laptop, smartphone, tablet, electronic reader, personal digital assistant, smart watch, various types of mobile devices, or other type of computing device capable of communication over network 140). User device 130 may be communicatively coupled to network 130 by way of the network interface. User device 130 may be coupled either directly to network 140 or through any number of intermediate servers, gateways, or other network devices. User device 130 may be configured to access data from external or remote storage media, such as memory cards or disk drives as may be appropriate in the case of downloaded services. User device 130 may include standard hardware computing components such as network and media interfaces, non-transitory computer-readable storage (memory), and one or more processors for executing instructions that may be stored in memory. User device 130 may be able to carry out various operations described herein by downloading and installing a software application (e.g., a smartphone application downloaded from an application store such as the App Store available on iPhone® and iPad®). Such a software application may allow a user (e.g., patient themselves, medical, dental, other health professional, researcher, or related assistants) to associate the application with monitoring device 110 and/or server 120.

Network 140 may be implemented as a private network, a public network, an intranet, a wide area network, the Internet, a local area network, or any suitable combination of the foregoing. Network 140 may be a local area network (LAN), which may be communicatively coupled to a wide area network (WAN) such as the Internet. The Internet is a broad network of interconnected computers and servers allowing for the transmission and exchange of Internet Protocol (IP) data between users connected through a network service provider. Examples of network service providers are the public switched telephone network, a cable service provider, a provider of digital subscriber line (DSL) services, or a satellite service provider.

FIG. 2 illustrates an exemplary oral health monitoring apparatus 200. Oral health monitoring apparatus 200 may include a mouthpiece housing 210, a pH sensor 220, and a wireless communication interface 230. Mouthpiece housing 210 may be fitted to a mouth of a patient, thus enabling the patient to comfortably wear apparatus 200 even for durations in which long-term oral health monitoring is desired. Sensor 220, which may be disposed inside or outside mouthpiece housing 210, may capture pH measurements of saliva in the mouth of the patient. The pH measurements may be captured over a predetermined time interval. Wireless communication interface 230, which may likewise be disposed inside or outside mouthpiece housing 210, sends the captured pH measurements to a server over a wireless communication network (e.g., network 140 of FIG. 1). As depicted in FIG. 2, mouthpiece housing 210 is fitted to a mouth of a patient and pH sensor 220 and wireless communication interface 230 are both disposed inside mouthpiece housing 210. Persons of ordinary skill in the art will readily appreciate, however, that other possible embodiments and arrangements are possible and suggested by the present disclosure.

Oral health monitoring apparatus 200 may include components that allow for wireless operation and communication, synchronization with remote devices, databases, servers, and user interfaces. Wireless communication interface 230, for instance, may be or may communicate with Bluetooth™ transmitters and receivers to communicate wirelessly with a nearby mobile device (e.g., pager, mobile phone, tablet, other computing device capable of communicating wirelessly), which may in turn transfer the information to a server or database for long-term storage.

In some embodiments, oral health monitoring apparatus 200 may be used without mouthpiece housing 210 and may measure the pH of samples outside the mouth. In such instances, pH sensor 220 may be structured as a stylus, probe, tongue depressor, or the like. Apparatus 200 with such a structure may be used to test samples that have already been provided or extracted from the human body. As with embodiments featuring mouthpiece housing 210, the captured pH measurements may be transferred wirelessly (e.g., via Wi-Fi, Bluetooth™, 3G, and other wireless protocols) to a separate computing device (e.g., mobile device, computing device, server, or the like) for storage, analysis, comparison, and other uses.

Oral health monitoring apparatus 200 may capture of a consistent flow of data regarding samples over a period of time. For example, embodiments featuring mouthpiece housing 210 may be used to collect saliva pH measurements overnight (e.g., 6-12 hours) or several days. Meanwhile, measurements regarding other health indicators (and the time the measurements were taken) may also be entered into a computing device (e.g., a mobile device, computing device, server, or the like). Once the pH measurements are received, such measurements may be plotted in a graph or chart over time, as well as compared to the other indicators. Such visual or graphical representations may therefore reveal various correlations between pH and one or more of the other health indicators. Such correlations may be used to research various conditions and determine the predictive value such non-invasive (e.g., saliva sampling) tests may have for various health conditions. For example, certain ranges of saliva pH may be correlated (along with other symptoms) to various inflammatory or other systemic conditions. The correlations may be used to formulate predictions and eventual diagnoses regarding various diseases or health conditions.

In an exemplary embodiment, a pH level of a sample from a particular patient may be tested by the patient, by a medical or dental professional, or other administrator. As discussed above, the pH level may be determined over a period of time by placing oral health monitoring apparatus 200 in the mouth of the patient and allowing pH sensor 220 to capture pH measurements. The period of time may be as short as a few seconds or may span multiple days. During the longer time spans, the patient may wear oral health monitoring apparatus 200 mostly continually or may put oral health monitoring apparatus 200 in the mouth at designated, but shorter periods of time. Each pH measurement may be stored in association with the time at which the measurement was captured. The time may be entered, sent, or otherwise correlated to the associated pH measurement. Likewise, time information may also be captured or entered for other measurements of other health indicators. Such pH and other measurements may be tracked over time, which allows for generation of reports and graphs indicating trends in pH and the other health indicators, as well as any correlations. As a variety of different types of samples (e.g., saliva, urine) may be tested, the reports may further track the pH trends by type.

Oral health monitoring apparatus 200 may send pH measurements continually over the period of time that it is in the mouth of the patient. In some embodiments, additional sensor and control functions may further allow for intelligent decisions related to the capture, communication, and analysis of data at oral health monitoring apparatus 200 itself. The pH measurements may be sent to another device (e.g., a recipient device) for storage and/or analysis. The pH measurements may be sent in real-time or may be sent in batches. In either case, each pH measurement may be associated with a time of capture so as to reveal any changes (or lack thereof) over time.

In addition, other measurements regarding other health indicators may also be entered into or received by the recipient device. Such measurements may be entered by the user, sent from another database/device, or captured by other sensor devices associated with the user. Such devices may be or may be associated with any type of server or other computing device as is known in the art, including standard hardware computing components such as network and media interfaces, non-transitory computer-readable storage (memory), and processors for executing instructions or accessing information that may be stored in memory. The functionalities of multiple servers may be integrated into a single server. Any of the aforementioned servers (or an integrated server) may take on certain client-side, cloud, cache, or proxy server characteristics. These characteristics may depend on the particular network placement of the server or certain configurations of the server. One common system used by service providers is the Dentrix® patient management system, which may be integrated with embodiments of the present invention. Through such integration, the health professional may allow certain records from their Dentrix® system to synchronize with the user accounts for their patients.

The pH and other measurements may be collected and used to generate a visual or graphical chart regarding such measurements over time. Such a chart may be displayed on an associated display screen or sent to another device with a display screen (e.g., user device 130 of FIG. 1). Further, various analyses may be performed on such charts to reveal trends, correlations, and statistical significances regarding the same. Such information may be packaged along with the visual or graphical data into a report regarding the patient, which may be sent to the patient or to a designated health care or research professional.

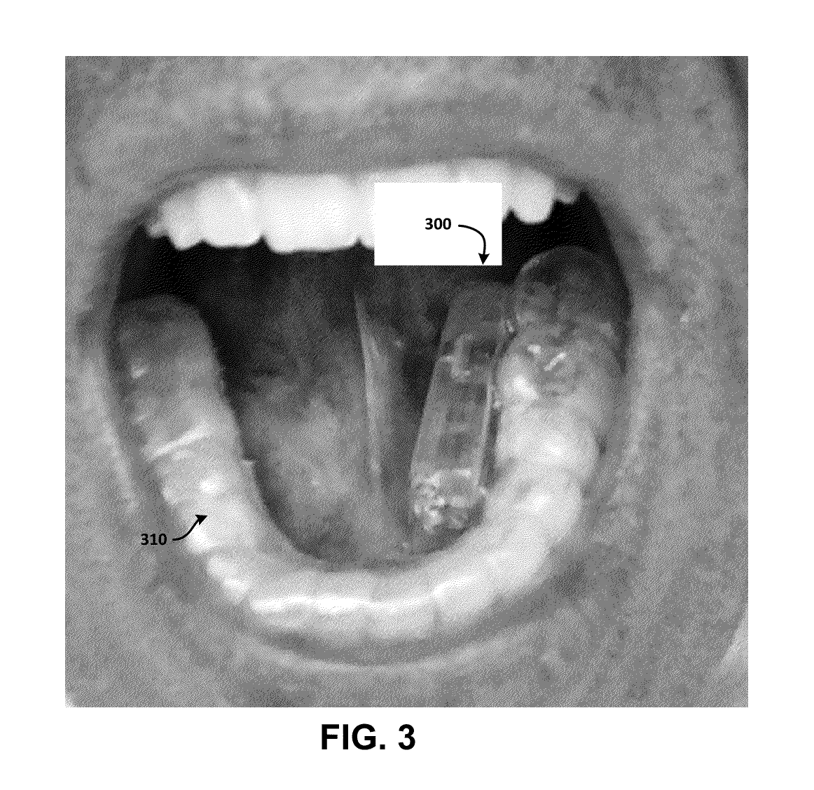

FIG. 3 illustrates an exemplary oral health monitoring apparatus 300 worn in a mouth of a patient. As illustrated in FIG. 3, mouthpiece housing 310 may be a clear or translucent housing that may be fitted to a mouth (e.g., teeth) of a patient.

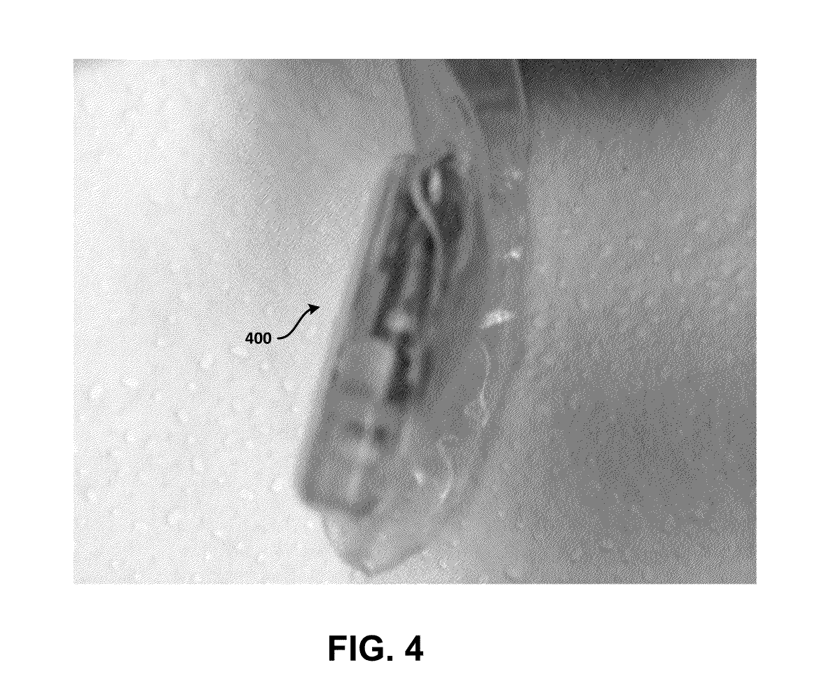

FIG. 4 illustrates an exemplary pH sensor 400 that may be implemented in the context of the oral health monitoring apparatus described herein. As noted above, pH sensor 400 may measure a pH level of saliva or other fluids found in a mouth of a patient. Sensor 400, along with any accompany components necessary for its functionality, may be coupled to a battery or other power source. The battery or other power source may be rechargeable (e.g., by placing the battery or other power source proximate to a charging platform or base).

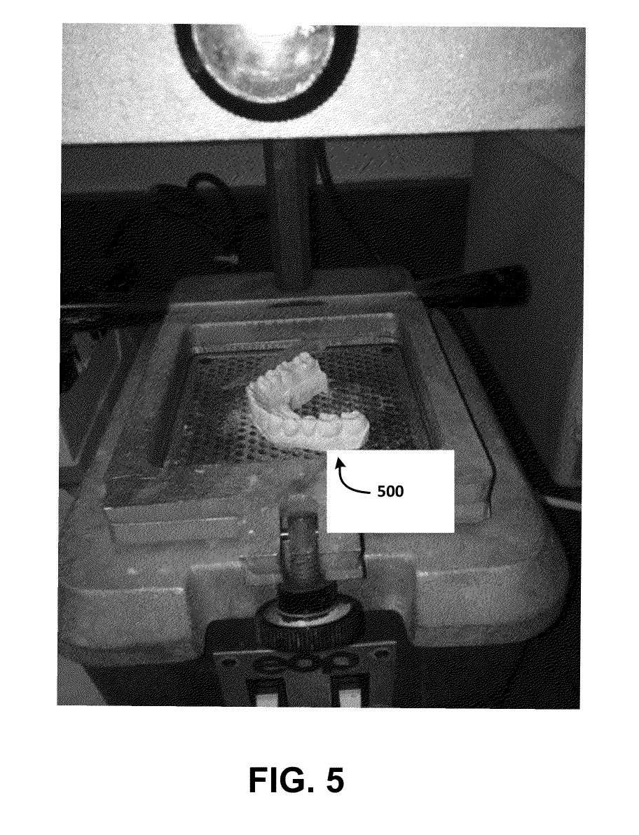

FIG. 5 illustrates an exemplary mold 500 that may be used to create a mouthpiece housing (e.g., mouthpiece housing 210 of FIG. 3). As discussed above, mouthpiece housing 210 may be fitted to a mouth of a patient, thus enabling the patient to comfortably wear the apparatus even for long durations.

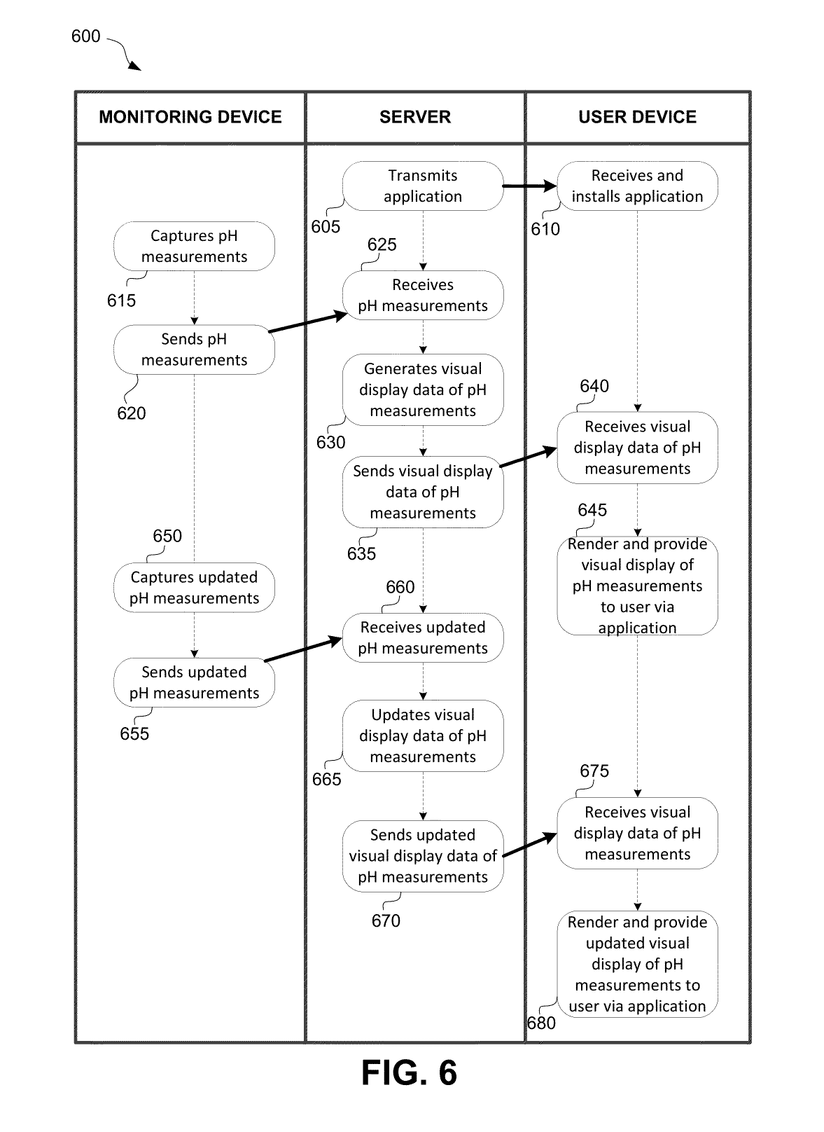

FIG. 6 illustrates an exemplary operational flow 600 of an exemplary oral health monitoring system. At block 605, a server of the oral health monitoring system (e.g., server 120 depicted in FIG. 1) may transmit a downloadable software application to a user device (e.g., user device 130 of FIG. 1). The downloadable software application, as noted above, may allow the user device to associate with a monitoring device (e.g., monitoring device 110 of FIG. 1) worn by a patient. The user may be the patient (e.g., in the case of self-monitoring) or a different person, such as a care provider. The server may transmit the downloadable software application in response to a request received from the user device over a communications network by which the server, user device, and monitoring device are all communicatively coupled (as shown, for example, in FIG. 1). Although FIG. 6 illustrates that the server receiving pH measurements from the monitoring device is the same server that transmits the downloadable application to the user device, persons of ordinary skill in the art will readily appreciate that the foregoing tasks may be distributed amongst multiple servers. The application may be transmitted, for instance, by a third-party server associated with an application database such as the App Store available on iPhone® and iPad®. At block 610, the user device may receive and install the software application. The user device may then be configured to associate itself with the monitoring device.

At block 615, the monitoring device, which may include a mouthpiece housing, may capture the plurality of pH measurements of saliva by way of a pH sensor. The monitoring device may capture the pH measurements over a predetermined time interval. The pH sensor may be disposed inside or outside the mouthpiece housing. The mouthpiece housing may be fitted to the mouth of the patient. The monitoring device may also include a wireless communication interface that communicates with the server over the communications network. Like the pH sensor, the wireless communication interface may be disposed inside or outside the mouthpiece housing.

At block 620, the monitoring device may send the pH measurements to the server over the communication network. The server may receive the pH measurements at block 625 and, after doing so, may generate a visual display of the pH measurements over time, which may include generating visual display data representing the pH measurements over time. At block 635, the server may send the visual display data representing the pH measurements to the user device. The user device may receive the visual display data at block 640 and, at block 645, may render and provide a virtual display of the pH measurements to the user by way of the downloaded application. In some embodiments, the visual display data may be rendered and displayed directly by the server or associated computing device.

At block 650, the monitoring device may continue capture updated pH measurements by way of the pH sensor. The monitoring device may send the updated pH measurements over the network to the server at block 660. At block 665, the server may update the visual display data represented the updated pH measurements. The server may then, at block 670, send the updated visual display data to the user device. The user device may receive the updated visual display data at block 675 and, at block 680, may render and provide the updated virtual display of the pH measurements to the user by way of the downloaded application.

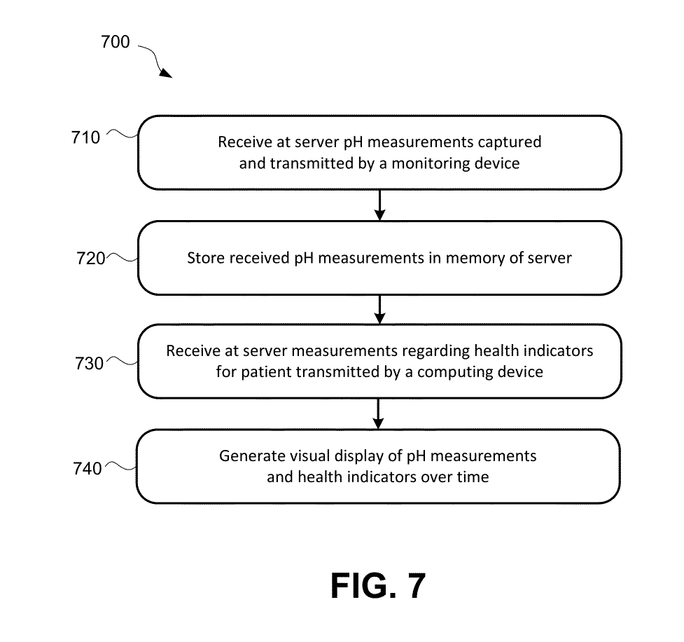

FIG. 7 illustrates an exemplary computer-implemented method 700 of monitoring oral health. Method 700 may include, at block 710, receiving pH measurements at a server. The measurements may be pH measurements of saliva in a mouth of a patient and may have been captured over a predetermined time interval by a monitoring device. The pH measurements may be received by the server over a wireless communication network and may be received from a monitoring device. At block 720, the method may include storing the received pH measurements in memory of the server. The method may also include, at block 730, receiving measurements regarding health indicators for the patient via a communication interface of the server. Each health indicator measurement may be associated with a time. The method may further include executing instructions stored in memory of the server. Upon execution of the instructions by a processor of the server at block 740, the method may include generating a visual display of the pH measurements over time. The visual display may further include the health indicator measurements over time.

Method 700 may be embodied as executable instructions in a non-transitory computer-readable storage medium, including but not limited to a CD, DVD, or non-volatile memory such as a hard drive. The instructions of the storage medium may be executed by a processor (or processors) to cause various hardware components of a computing device hosting or otherwise accessing the storage medium to effectuate the method. The steps described herein (and the order thereof) are exemplary and may include various alternatives, equivalents, or derivations thereof including but not limited to the order of execution of the same.

Non-transitory computer-readable storage media refer to any medium or media that participate in providing instructions to a central processing unit (CPU) for execution. Such media can take many forms, including, but not limited to, non-volatile and volatile media such as optical or magnetic disks and dynamic memory, respectively. Common forms of non-transitory computer-readable media include, for example, a floppy disk, a flexible disk, a hard disk, magnetic tape, any other magnetic medium, a CD-ROM disk, digital video disk (DVD), any other optical medium, RAM, PROM, EPROM, a FLASHEPROM, and any other memory chip or cartridge.

Various forms of transmission media may be involved in carrying one or more sequences of one or more instructions to a CPU for execution. A bus carries the data to system RAM, from which a CPU retrieves and executes the instructions. The instructions received by system RAM can optionally be stored on a fixed disk either before or after execution by a CPU. Various forms of storage may likewise be implemented as well as the necessary network interfaces and network topologies to implement the same.

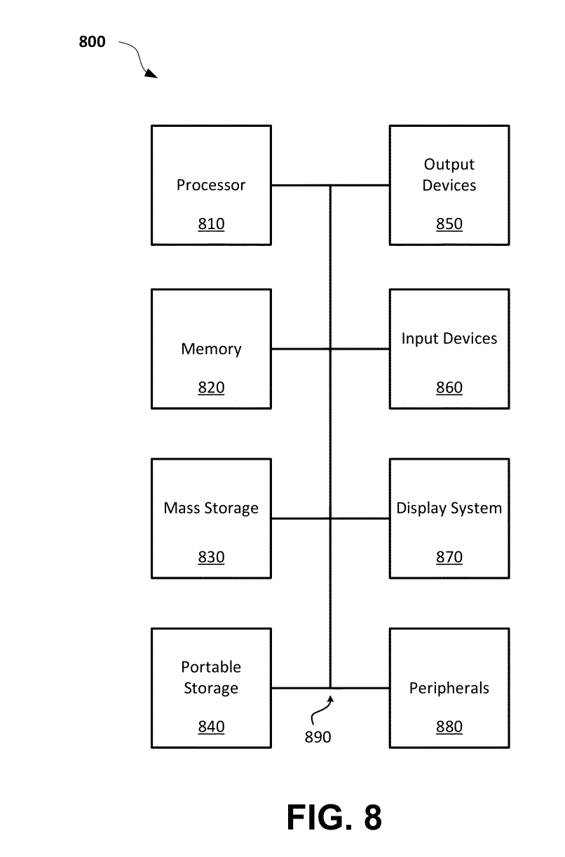

FIG. 8 illustrates an exemplary system 800 for implementing a computing device. The computing system 800 of FIG. 8 may be implemented in the context of client 110, client proxy 120, a server proxy 140, or server 150 of FIG. 1. The computing system of FIG. 8 may include one or more processors 810 and memory 820. Main memory 820 may store, in part, instructions and data for execution by processor 810. Main memory 820 may store the executable code when in operation. Computing system 800 may further include a mass storage device 830, a portable storage medium drive 840, output devices 850, user input devices 860, a graphics display system 870, and peripheral devices 880.

The components shown in FIG. 8 are depicted as being connected via a single bus 890. The components may alternatively be connected through one or more data transport means. Processor 810 and main memory 820, for example, may be connected via a local microprocessor bus. Mass storage device 830, peripheral device(s) 880, portable storage device 840, and display system 870 may be connected via one or more input/output buses.

Mass storage device 830, which may be implemented with a magnetic disk drive or an optical disk drive, may be a non-volatile storage device for storing data and instructions for use by processor 810. Mass storage device 830 may store system software for implementing embodiments of the solution described herein for purposes of loading the software into main memory 820.

Portable storage device 840 may operate in conjunction with a portable non-volatile storage medium, such as a compact disk or digital video disc, to input and output data and code to and from computer system 800. The system software for implementing embodiments of the present solution may be stored on such a portable medium and input to computer system 800 via portable storage device 840.

Input devices 860 may provide a portion of a user interface. Input devices 860 may include an alpha-numeric keypad, such as a keyboard, touch screen, or touchpad, for inputting alpha-numeric and other information, or a pointing device, such as a mouse, a trackball, stylus, or cursor direction keys. Additionally, system 800 may include output devices 850, such as speakers, printers, network interfaces, monitors, and the like.

Display system 870 may include a liquid crystal display or other suitable display device. Display system 870 may receive textual and graphical information and may process the information for output to the display device.

Peripherals 880 may include any type of computer support device to add additional functionality to computer system 800. Peripheral device 880 could be, for example, a modem or a router.

The components illustrated in computer system 800 of FIG. 8 are those typically found in computer systems that may be suitable for use with embodiments of the present solution. The depiction of such components is not intended to be exhaustive in nature, but is rather intended to represent a broad category of computer components that are well known in the art. Thus, system 800 may be a desktop computer, workstation, server, mainframe computer, laptop, tablet, smartphone or other mobile or hand-held computing device, or any other suitable computing device. Computer system 800 may also include various bus configurations, networked platforms, multi-processor platforms, and the like. Various operating systems may be used, such as a UNIX™ operating system, a LINUX™ operating system, a WINDOWS™ operating system, a MACINTOSH™ operating system, a PALM™ operating system, and other suitable operating systems.

While various embodiments have been described above, it should be understood that they have been presented by way of example only, and not limitation. The descriptions are not intended to limit the scope of the invention to the particular forms set forth herein. Thus, the breadth and scope of a preferred embodiment should not be limited by any of the above-described exemplary embodiments. It should be understood that the above description is illustrative and not restrictive. To the contrary, the present descriptions are intended to cover such alternatives, modifications, and equivalents as may be included within the spirit and scope of the invention as defined by the appended claims and otherwise appreciated by one of ordinary skill in the art. The scope of the invention should, therefore, be determined not with reference to the above description, but instead should be determined with reference to the appended claims along with their full scope of equivalents.

What is claimed is:

1. An oral health monitoring apparatus, the apparatus comprising:
a mouthpiece housing;
a pH sensor that captures a plurality of pH measurements of saliva in a mouth of a patient; and
a wireless communication interface that sends the captured plurality of pH measurements over a wireless communication network to a server.

2. The apparatus of claim 1, wherein the mouthpiece housing is fitted to the mouth of the patient.

3. The apparatus of claim 1, wherein the pH sensor is disposed in the mouthpiece housing.

4. The apparatus of claim 1, wherein the wireless communication interface is disposed in the mouthpiece housing.

5. The apparatus of claim 1, wherein the received pH measurements are captured over a predetermined time interval.

6. An oral health monitoring system, the system comprising:
a monitoring device that captures a plurality of pH measurements of saliva in a mouth of a patient; and
a server that receives the pH measurements from the monitoring device over a wireless communication network.

7. The system of claim 6, wherein the server generates a visual display of the pH measurements over time.

8. The system of claim 7, wherein the server transmits the visual display of the PH measurements over time to a mobile device of a user.

9. The system of claim 6, wherein the monitoring device captures the plurality of pH measurements of saliva by way of a pH sensor.

10. The system of claim 6, wherein the monitoring device includes a mouthpiece housing.

11. The system of claim 6, wherein the mouthpiece housing is fitted to the mouth of the patient.

12. The system of claim 9, wherein the pH sensor is disposed in the mouthpiece housing.

13. The system of claim 6, wherein the monitoring device includes a wireless communication interface that communicates with the server over the wireless communications network.

14. The system of claim 13, wherein the wireless communication interface is disposed in the mouthpiece housing.

15. The system of claim 6, wherein the monitoring device captures the pH measurements over a predetermined time interval.

16. A method of monitoring oral health, the method comprising:
receiving at a server a plurality of pH measurements for saliva in a mouth of a patient,
storing the received pH measurements in memory of the server;
receiving a plurality of measurements regarding a plurality of health indicators for the patient via a communication interface of the server; and
executing instructions stored in memory, wherein execution of the instructions by a processor of the server generates a visual display of the pH measurements over time.

17. The method of claim 16, wherein the received pH measurements are captured over a predetermined time interval.

18. The method of claim 16, wherein the received pH measurements are received by the server over a wireless communication network.

19. The method of claim 16, wherein each health indicator measurement is associated with a time.

20. The method of claim 16, wherein the visual display further includes the health indicator measurements over time.
